(19) United States
(12) Patent Application Publication
(10) Pub. No.: US 2025/0271396 A1
(43) Pub. Date: Aug. 28, 2025

(54) HYBRID PROBE AND METHOD FOR REAL-TIME MICROSTRUCTURE IDENTIFICATION BY NON-DESTRUCTIVE MAGNETIC TESTING

(71) Applicants: Petróleo Brasileiro S.A. – Petrobras, Rio de Janeiro (BR); Universidade Federal do Rio de Janeiro — UFRJ, Rio de Janeiro (BR)

(72) Inventors: Luiz Henrique DE ALMEIDA, Rio de Janeiro (BR); Carlos Bruno ECKSTEIN, Rio de Janeiro (BR); Iane DE ARAÚJO SOARES, Rio de Janeiro (BR); Lucas Braga CAMPOS, Rio de Janeiro (BR); Clara Johanna PACHECO, Rio de Janeiro (BR); Laudemiro NOGUEIRA JUNIOR, Rio de Janeiro (BR); Daniel Mendes FERNANDES, Rio de Janeiro (BR); Mónica Patricia Arenas CORREA, Esch-sur-Alzette (LU); Gabriela Ribeiro PEREIRA, Rio de Janeiro (BR)

(21) Appl. No.: 19/056,243

(22) Filed: Feb. 18, 2025

(30) Foreign Application Priority Data
Feb. 22, 2024 (BR) 1020240034449

(51) Int. Cl. G01N 27/90; G01N 27/904; G01N 33/2045

(52) U.S. Cl. CPC G01N 27/9006; G01N 27/904; G01N 33/2045

(57) ABSTRACT

The present invention discloses a device and a method for inspecting furnace catalyst tubes. The device comprises two eddy current sensors consisting of an eddy current excitation coil and a Hall effect sensor, wherein the second of these sensors also includes a yoke where permanent magnets are positioned on its sides. The permanent magnets cause an external surface layer of oxides on the surface of the tubes to be magnetically saturated, allowing the eddy currents generated by the second sensor to penetrate the interior of the tube wall. In this way, eddy current data are obtained from the interior of the tube wall by the second sensor and from the tube surface by the first sensor. These data are processed by an analyzing element that incorporates an artificial intelligence previously trained to determine the aging state of the tube based on the data obtained by the sensors. In this way, a non-destructive and real-time analysis of the aging state of the tube is achieved. The hybrid probe can be used manually to determine the aging state of a specific point on a tube or in automated inspection, in which an autonomous mobile device guides the probe along the tube, allowing real-time determination of the longitudinal profile of the aging states.

### CROSS-REFERENCE

This application claims priority to Brazilian Patent Application No. 1020240034449, filed Feb. 22, 2024, which is incorporated herein in its entirety by reference thereto.

### FIELD OF THE INVENTION

The present invention falls within the field of material engineering. More specifically, the present invention relates to methods and devices for inspecting in real time the microstructures of paramagnetic metallic materials.

### BACKGROUND OF THE INVENTION

The furnaces used in steam reforming processes use catalyst tubes made of high-temperature steels. During operation, the wall of these tubes is subjected to temperatures that occasionally vary in the range of 600 to 1000° C. Over time, these tubes undergo microstructural changes and accumulate damage and need to be replaced to ensure operational safety. At these temperatures, the predominant mechanism of damage is due to the creep phenomenon, which is characterized by causing cumulative deformations in the material that eventually lead to its rupture.

Currently, in inspection procedures during maintenance shutdowns, when it is desired to determine the aging state of the steel, two approaches are used, both destructive: metallographic replication or optical metallography on a sample removed from the tube.

Metallographic replication is generally performed in the field and consists of roughing a region of approximately 15 cm2 on the surface of the tube followed by metallographic polishing. After obtaining a flat and mirror-like surface, a chemical attack is carried out which, due to the differential corrosion of the several microconstituents of the material, generates a relief. In the final preparation stage, an acetate film is then applied which, when removed, has on its surface the “negative” of the relief caused by the corrosion. This film is taken to the optical microscope where the microstructure is revealed to be classified by comparison with a pattern of micrographs in one of the six microstructural aging states, characteristic of the catalyst tubes of steam reforming furnaces subjected to service temperatures.

This technique is time-consuming and requires the construction of scaffolding to access the point to be inspected, which limits, for practical reasons, the possibility of inspection to a few units at each stop.

Optical metallography requires the removal of the tube, which is subsequently cut at one or more points to remove samples. These are embedded in resin and undergo a polishing and chemical etching process similar to those performed in the metallographic replication. The sample is then taken to an optical microscope where the microstructure is observed and classified by comparison with a pattern of micrographs in one of the six characteristic aging states.

The major disadvantage of this technique is the need to remove one or more tubes from the furnace, which will have to be replaced, resulting in high costs, in addition to impacting other maintenance activities and significantly extending the maintenance shutdown time.

The development of non-destructive techniques that allow a prediction of the remaining life of these tubes is essential to guarantee the operational safety and profitability of the plant. Premature replacement of tubes has a major financial impact on the operating costs of the unit, and the occurrence of a failure during operation represents a risk of material and human losses, in addition to a major economic loss due to loss of profit.

### STATE OF THE ART

The document “Eddy Current Testing: Basics (Purna Chandra Rao Bhagi; Non-destructive Evaluation Division, Metallurgy and Materials Group Indira Gandhi Center for Atomic Research, Kalpakkam—603 102, TN, India. volume 10, issue 3, December 2011)”, discloses principles of testing and sensing based on eddy currents. This document points out, among others, that eddy currents are sensitive to changes in microstructure and stresses, which alter the electrical conductivity and magnetic permeability of the material. The detection and sizing of incipient surface defects as well as subsurface defects, changes in microstructures and accumulated plastic deformation, stress or damage, e.g. prior to the formation of microcracks, etc. Eddy currents also generate a secondary magnetic field that can be detected using a separate receiver coil or a solid-state field detection sensor. Typically, eddy current measurements for microstructure characterization are location-based. Absolute probes are generally used, and the analysis is based on the interpretation of the impedance plane signal. Reference standards with known electrical conductivity and magnetic permeability from heat-treated specimens at different aging conditions are used to establish the calibration chart.

The patent BR 112015016852-3 B2, published on Jul. 11, 2017, entitled “Sistema magnético de medição para um detector de falhas com magnetização longitudinal” (Magnetic measuring system for a flaw detector with longitudinal magnetization), relates to the use of a magnetic field for detecting defects, with devices for non-destructive testing of pipelines, and to a magnetic measuring system for a flaw detector in the line. A magnetic measuring system for a flaw detector having longitudinal magnetization is based on combined sensor units and makes it possible to detect flaws on the basis of a stray field that is formed where flaws are present, to determine whether a flaw refers to the inner wall of a pipeline, and to image distortions in the shape of the inner wall of the pipeline, related to dents, transverse seams, etc. Combined sensor units are installed on a cylindrical magnetic core of a single-section flaw detector by means of a ring of movable supports between two rings of magnets with opposite polarity and elements that transmit magnetic flux to the inner wall of the conduit. A combined sensor unit consists of eddy current sensors and Hall sensors to measure the transverse component of the induction of the magnetic field, and Hall sensors to measure the longitudinal component of the magnetic field. An eddy current sensor, in its turn, consists of two induction coils that are combined in a module and positioned coaxially, one above the other. One of the induction coils acts as a reference coil, since it is located in a shielded compartment that is isolated from the external environment in the direction of the electromagnetic current, and the inductance of the second, non-isolated, inductance coil is compared with the inductance of the reference coil.

The application EP 1564551 A1, published on Aug. 17, 2005, entitled “Non-destructive method for the detection of creep damage in ferromagnetic parts with a device consisting of an eddy current coil and a Hall sensor”, discloses a non-destructive method for the detection of creep damage in power plant components made of ferromagnetic steels, such as gas turbine rotors. More specifically, it discloses a method for measuring magnetic and conductive properties using a specific sensor design that adapts a system called 3MA (micromagnetic, multiparameter, microstructure and stress analysis). The device must be calibrated by performing a specific procedure. The steps of manufacturing a probe consisting of an eddy current coil and a Hall sensor with a magnetization yoke adapted to the site of interest and manufacturing curved and flat calibration specimens with defined creep deformation and with different surface conditions. Suitable creep laws and finite element (FE) methods were applied to calculate the creep loading conditions required to produce the calibration sample. Once the system is calibrated, it can be used to measure creep deformation in ferromagnetic steel components made of the same material and curvature as the calibration sample.

### SUMMARY OF THE INVENTION

The present invention consists of a methodology based on the eddy current technique applied to a paramagnetic material that has a surface layer of magnetic oxides. A hybrid probe was developed to identify the aging states, in addition to a classifier generated by machine training (Support Vector Machine—SVM) to classify, in real time, these aging states. The hybrid probe consists of two sensors. Each of these sensors consists of a coil and a Hall sensor. In addition, as an integral part of the architecture of the developed hybrid probe, one of the sensors has a yoke with permanent magnets at the ends, whose function is to magnetically saturate the external surface layer of oxide in the tube. The hybrid probe can be used manually to determine the aging state of a specific point of a tube or in automated inspection, in which an autonomous mobile device guides the probe along the tube, allowing the determination of the longitudinal profile of the aging states.

### BRIEF DESCRIPTION OF THE DRAWINGS

The present invention will be described below with reference to the typical embodiments thereof, and also with reference to the attached drawings.

FIG. 1 is a representation of the sensors according to the present invention.

FIG. 2 is a representation of a mechanical module that supports the sensors according to the present invention.

FIG. 3 is a representation of the sensor assembly in the mechanical module.

FIG. 4 is a representation of the assembly formed by the Yoke and the permanent magnets.

FIG. 5 is a representation of the mechanical module positioned in a tube.

FIG. 6 is a representation of the training matrix for the formation of the classifier 5.

### DETAILED DESCRIPTION OF THE INVENTION

Specific embodiments of the present disclosure are described below. In an effort to provide a concise description of these embodiments, all features of an actual implementation may not be described in the specification. It should be appreciated that in the development of any actual implementation, as in any engineering or design project, numerous specific implementation decisions must be made to achieve the specific objectives of the developers, such as compliance with system and business-related constraints, which may vary from one implementation to another. Furthermore, it should be appreciated that such a development effort may be complex and time-consuming but would nevertheless be a routine design and manufacturing undertaking for those of ordinary skill having the benefit of this disclosure.

The catalyst tubes, installed inside the radiation chamber of the steam reforming furnaces for hydrogen production, are manufactured from HP class austenitic stainless steel, heat resistant and centrifugally cast. A characteristic of HP steel is that when operating at temperatures above 600° C., even for times of the order of a few thousand hours, which are relatively short when compared to the time they are kept in operation, it undergoes an irreversible transformation in its microstructure that is directly related to the temperature to which they were exposed. Thus, once the microstructure of a given region of the tube is known, it is possible to determine a temperature range to which this region was subjected during operation, which is strategic information in the methodology for predicting the remaining life. These microstructural characteristics are classified into six groups that correspond to six temperature ranges and are identified as aging states from I to VI. Non-destructive identification of aging states is important for predicting the useful life of these tubes. In the current scenario, the methodology for assessing the condition of the material by measuring the diameter of some sections of the tubes in the reforming furnace is very uncertain.

Another characteristic of HP steel is that it is a paramagnetic material. The inventors noticed that, during the operation of the furnaces, an external surface layer of oxides is formed on the tubes due to exposure to combustion gases from the furnace at high temperatures. This external surface layer of oxides is composed of different oxides depending on the temperature to which the steel was exposed and has a characteristic ferromagnetic response.

It is known that eddy currents are intimately affected by changes in the metallic microstructure of materials, and HP steel is no different. There are some examples in the state of the art of devices and methods that use eddy currents to observe the microstructure of materials. The inventors noted that during the operation of the furnaces, a surface layer of oxides is formed on the external surface of the tubes due to exposure to combustion gases from the furnace at high temperatures.

The external surface layer of oxides is composed of different oxides and has a ferromagnetic response. Consequently, the density of eddy currents is concentrated on this surface due to the high magnetic permeability of this layer. Therefore, an external magnetic field was applied to cause partial magnetic saturation of the external surface layer of oxides, the eddy currents were able to reach the region of the center of the tube wall.

In view of these facts, the inventors developed a device and associated method to overcome the deficiencies of the state of the art.

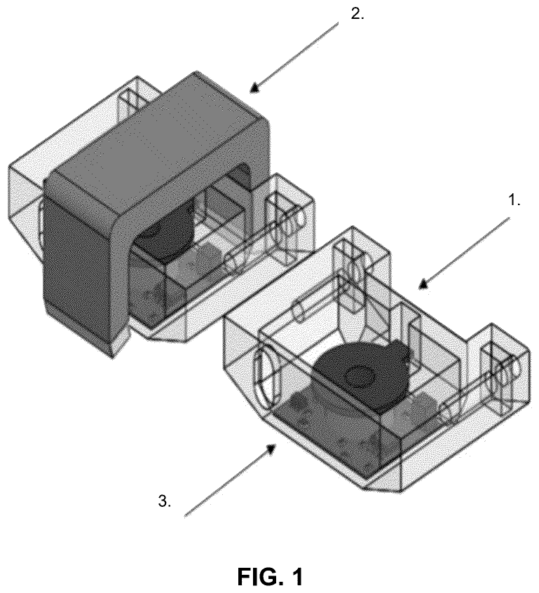

In a first aspect, the present invention consists of a probe comprising two hybrid sensors as can be seen in FIG. 1. The first sensor 1 is formed by an eddy current coil and a Hall effect sensor as already well described in the art. Since the external surface of the tube has a strong magnetic response due to the oxide layer, the sensor 1 characterizes only this region. The second sensor 2 is also formed by an eddy current coil and a Hall effect sensor plus a yoke 14 with permanent magnets 15 at its ends. The magnets 15 are such that the magnetic field they generate through the tube causes a partial magnetic saturation of the external surface layer of oxides, inhibiting its magnetic response. In this way, the sensor 2 is capable of reading the inside of the tube wall.

To reduce mechanical vibration and improve the signal-to-noise ratio, sensors 1 and 2 are preferably contained, each one, in an encapsulation together with its respective electronic board 3 for amplification of the output signal of the Hall effect sensor. Each encapsulation is preferably filled with epoxy resin. Sensors 1 and 2 are connected to an electronic data acquisition board 4, responsible for powering the coil and acquiring signals emitted by sensors 1 and 2. Through the signals collected by electronic board 4, it is possible to create a classifier 5.

The classifier 5 determines the aging state of the HP steel pipe. The classifier 5 is a learning model that was created from the data collected by sensors 1 and 2, through supervised machine training SVM (Support Vector Machine). Advantageously, when receiving new data collected from sensors 1 and 2, the classifier 5 is able to automatically identify in real time the aging state of the analyzed pipe.

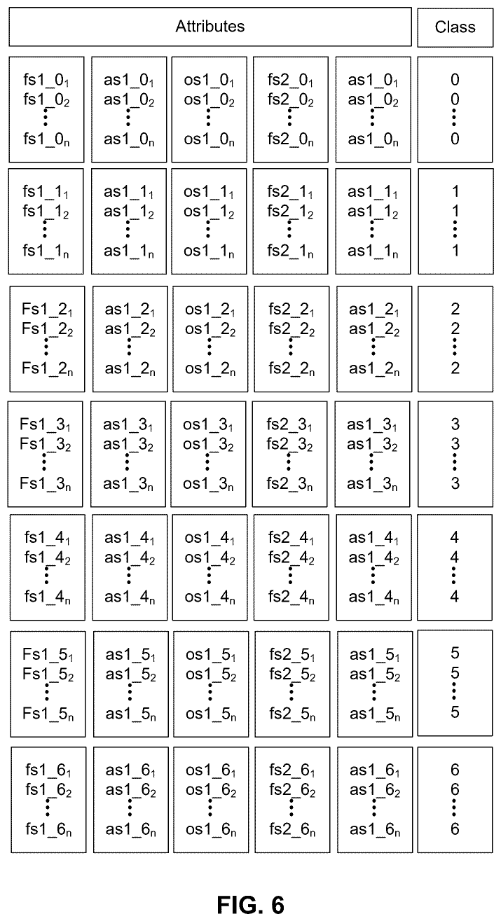

In the training of the classifier 5, pipe segments whose aging states were known were used. Previous studies show that both the outer oxide surface layer and the wall center have unique characteristics that define their aging state. Therefore, it is necessary to acquire information from these regions. For example, the outer oxide surface layer grows with the aging state. The probe of the invention was coupled to each pipe segment to collect data from the entire perimeter of the pipe. The phase f and amplitude A data from both sensors, 1 and 2, and the offset of sensor 1 were stored together with the aging states in a six-column matrix, exemplified in FIG. 6, where the first five columns contain the attributes (phase of sensor 1, amplitude of sensor 1, offset of sensor 1, phase of sensor 2, amplitude of sensor 2) and the last column names the aging state (class) of the respective pipe segment. The combination of these variables extracted from sensors 1 and 2 are used to create the classifier 5, which was trained using the Support Vector Machine (SVM) method, applying the “k-fold” cross-validation technique. This method consists of separating the collected data into classes using a hyperplane that has the greatest distance between them. However, the “k-fold” cross-validation technique verifies the classifier, that is, whether the model created has a good generalization capacity for new data. With k=10, the data is randomly divided into 10 subsets, with 9 subsets used for training and I subset for testing. In this case, the process is repeated 10 times. A person skilled in the art will realize that other machine learning methodologies are applicable without departing from the present invention.

Once trained and validated, the classifier 5 becomes capable of automatically identifying the aging state of a new pipe segment. By positioning sensors 1 and 2 on the outer layer of the tube and scanning, the data obtained by sensors 1 and 2 and their respective phases f, amplitudes A and the offset of sensor 1 are analyzed by classifier 5. The SVM evaluates the data acquired from these two regions, recognizing the patterns in the results as points in space in relation to what was found in the samples with the different aging states used in the training.

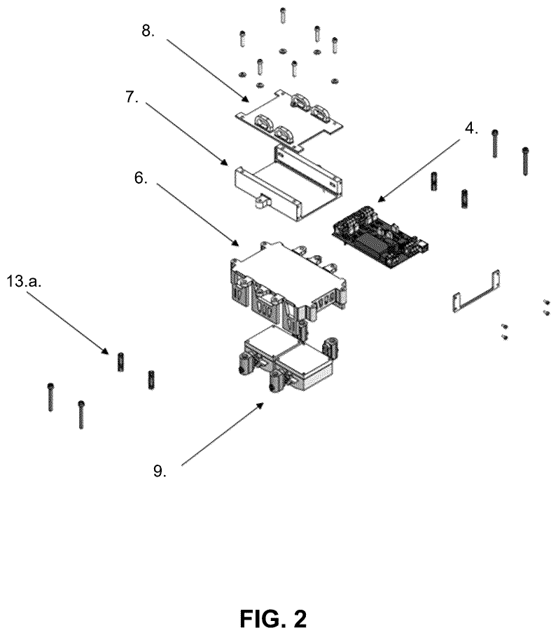

The sensors 1 and 2 are also preferably mounted on a mechanical module as exemplified in FIG. 2 to assist in coupling and moving the probe along the tube to be inspected. The exemplary and non-limiting mechanical module of FIG. 2 consists of a structural support 6, electronic board protection box 4, protection box cover 8, and two sensor assemblies 9.

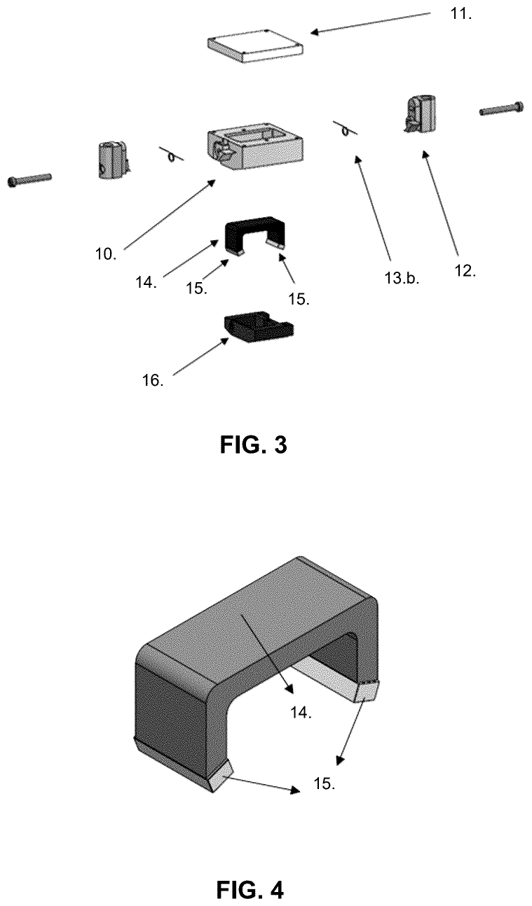

With reference to FIG. 3, each sensor assembly 9 is formed by each hybrid eddy current sensor 1 and 2, yoke 14 of sensor 2 inserted in its respective case 10 with cover for case 11 and mounted on base 16 of the case, base 12 for compression and torsion springs, and springs 13 (compression spring 13.a. and torsion spring 13.b). Each sensor 1 and 2 is mounted on support 6 and has two movements by the action of springs 13, which press the sensors against the surface of the tube.

FIG. 4 shows in more detail the yoke 14 with the permanent magnets 15 located at its ends.

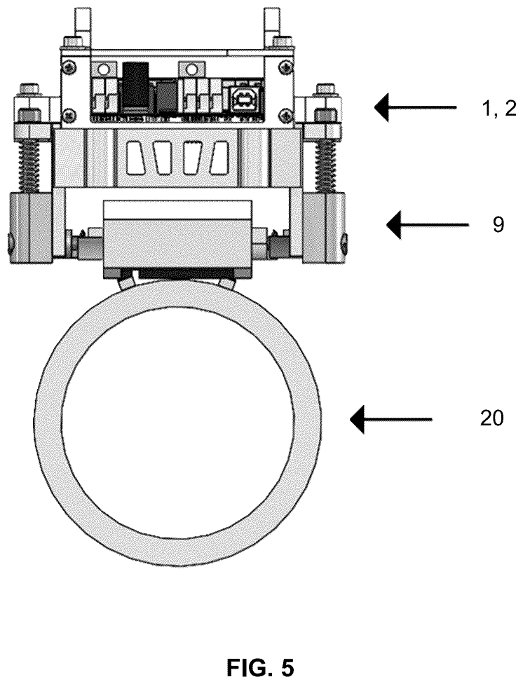

FIG. 5 shows the sensor device of the present invention fully assembled and positioned in a pipe 20 to be inspected.

According to the description above, the probe of the present invention is capable of performing non-destructive inspection of catalyst tubes to determine the aging state of these tubes. In addition, the probe of the present invention provides a profile of the aging states of the inspected tube in real time, which greatly reduces the downtime required for furnace maintenance.

The probe of the present invention, mounted on the mechanical module, can be moved along the surface of a tube to be inspected manually, but preferably this is done automatically to further speed up the inspection.

There are several ways to automate the movement of the mechanical module depending on the specific application. For example, the position of the tubes, whether the movement will be vertical, horizontal or both, and the space available to maneuver the mechanical module are factors that influence the choice of the movement device. However, preferably, the mechanical module is mounted on a device equipped with motorized tracks and fixed to the tube.

The power supply for both the probe and the movement device can be provided by batteries, or by an external wired source, or by a combination of both.

A second aspect of the invention is related to a pipe inspection method that uses the device disclosed above in the first aspect of the invention.

The method according to the second aspect of the present invention comprises the steps of:
1) positioning the hybrid probe as defined in the first aspect of the present invention in a pipe to be inspected, the hybrid probe comprising:
   a first sensor 1 comprising an eddy current coil, a Hall effect sensor, and an electronic board 3,
   a second sensor 2 comprising an eddy current coil, a Hall effect sensor, a yoke 14 with permanent magnets 15 at its ends, and an electronic board 3,
   an electronic data acquisition board 4 in communication with the first sensor 1 and with the second sensor 2 to receive data generated by the first sensor 1 and by the second sensor 2, and
   a classifier 5 in communication with the electronic data acquisition board 4 and incorporating an artificial intelligence to analyze the data obtained by the electronic data acquisition board 4;
2) supplying sensor 1 and sensor 2 in series to generate eddy currents in the tube to be inspected and sending the data obtained to the electronic data acquisition board 4;
3) forwarding the data acquired by the electronic data acquisition board 4 to the classifier 5; and
4) obtaining, by the classifier 5, an aging status of the tube based on the acquired data.

Optionally, the above method may comprise an additional step 5) of moving, manually or automatically, the hybrid probe along the length of the tube, thus generating an aging profile of the same.

The above method is preferably performed on furnace catalyst tubes, since such tubes have an external surface layer of oxides. As previously mentioned, this external surface layer of oxides interacts with the hybrid probe in such a way that the inspection performed by the first sensor 1 takes place on the surface of the tube and the inspection performed by the second sensor 2 takes place inside the tube wall since the permanent magnets 15 installed on the sides of the yoke 14 partially magnetically saturate the external surface layer of oxides, allowing the induced eddy currents to be located inside the tube wall.

The advantages of the device and method disclosed above according to the present invention will be evident to a person skilled in the art.

As it is a non-destructive technique, inspection with the developed hybrid probe does not cause any changes to the pipes, allowing immediate return to operation after maintenance shutdown. While the execution of each metallographic replication according to the state of the art, from the preparation of the pipe surface to its interpretation under the microscope, takes between 3 and 4 hours, the inspection of the same region with the probe of the present invention is done in real time.

As it is possible to perform the inspection with the sensor in motion, when installed in a device capable of moving it along the surface of the pipes, it allows the inspection of the entire length of a large number of pipes in each work shift.

Knowledge of the aging status of a large number of tubes, or even all of them, not only contributes to the assessment of their remaining life, but can also help equipment operators diagnose operational problems such as, for example, the non-uniform distribution of heat provided by the burners to the furnace tubes.

By contributing to a better prediction of the remaining life of furnace tubes, inspection with the probe of the present invention reduces the risk of premature tube replacement and tube failure during operation. Premature tube replacement has a financial impact due to the early capital outlay, and a failure during operation, in addition to the risk of material and human losses, causes a large economic loss due to loss of profit. The use of the technique described herein brings clear economic and safety gains to the units to which it is applied.

Although aspects of the present disclosure may be susceptible to various modifications and alternative forms, specific embodiments have been shown by way of example in the drawings and have been described in detail in this document. But it should be understood that the invention is not intended to be limited to the particular forms disclosed. Rather, the invention is intended to cover all modifications, equivalents and alternatives that fall within the scope of the invention as defined by the following appended claims.

1. A hybrid probe for real-time microstructure identification by non-destructive magnetic testing, comprising:
a first hybrid eddy current sensor comprising an eddy current coil, a Hall effect sensor, and an electronic board;
a second hybrid eddy current sensor comprising an eddy current coil, a Hall effect sensor, a yoke with permanent magnets at its ends, and an electronic board;
an electronic data acquisition board in communication with the first hybrid eddy current sensor and with the second hybrid eddy current sensor to receive data generated by the first hybrid eddy current sensor and by the second hybrid eddy current sensor; and
a classifier in communication with the electronic data acquisition board,
wherein the classifier is configured to incorporate artificial intelligence to analyze the data obtained by the electronic data acquisition board, and
wherein the classifier is configured to determine an aging state of a pipe to be inspected.

2. The hybrid probe of claim 1, further comprising a structural support, an electronic board protection box, a protection box cover and two sensor assemblies, each sensor assembly being formed by each hybrid eddy current sensor, the yoke of the second sensor inserted in a respective case with a cover for the case, base for compression and torsion springs and springs.

3. The hybrid probe of claim 2, wherein the structural support is mounted on an automated movement device.

4. A method for real-time microstructure identification by non-destructive magnetic testing for use with the hybrid probe of claim 1, comprising the steps of:
positioning the hybrid probe in a pipe to be inspected;
supplying in series the first sensor and the second sensor through the electronic data acquisition board to generate eddy currents in the pipe to be inspected and sending the data obtained to the electronic data acquisition board;
forwarding the data acquired by the electronic data acquisition board to the classifier; and
obtaining, by the classifier, an aging status of the pipe based on the acquired data.

5. The method of claim 4, further comprising the step of:
moving, manually or automatically, the hybrid probe along a length of the pipe to generate an aging profile of the same.

What is claimed is:

1. Hybrid probe for real-time microstructure identification by non-destructive magnetic testing, characterized by comprising:
a first hybrid eddy current sensor (1) comprising an eddy current coil, a Hall effect sensor, and an electronic board (3);
a second hybrid eddy current sensor (2) comprising an eddy current coil, a Hall effect sensor, a yoke (14) with permanent magnets (15) at its ends, and an electronic board (3);
an electronic data acquisition board (4) in communication with the first sensor (1) and with the second sensor (2) to receive data generated by the first sensor (1) and by the second sensor (2); and
a classifier (5) in communication with the electronic data acquisition board (4) and that incorporates artificial intelligence to analyze the data obtained by the electronic data acquisition board (4),
wherein the classifier (5) is configured to determine an aging state of a tube to be inspected.

2. Hybrid probe, according to claim 1, characterized by further comprising a structural support (6), an electronic board protection box (7), a protection box cover (8) and two sensor assemblies (9), each sensor assembly (9) being formed by each hybrid eddy current sensor (1) and (2), yoke (14) of the sensor (2) inserted in its respective case (10) with cover for the case (11), base for compression and torsion springs (12) and springs (13).

3. Hybrid probe, according to claim 2, characterized in that the structural support (6) is mounted on an automated movement device.

4. Method for real-time microstructure identification by non-destructive magnetic testing for use with the hybrid probe, as defined in any one of claims 1 to 3, characterized by comprising the steps of:
1) positioning the hybrid probe in a tube to be inspected;
2) supplying in series the hybrid sensor 1 and the hybrid sensor 2 through the electronic data acquisition board 4 to generate eddy currents in the pipe to be inspected and sending the data obtained to the electronic data acquisition board 4;
3) forwarding the data acquired by the electronic data acquisition board 4 to the classifier 5; and
4) obtaining, by the classifier 5, an aging status of the pipe based on the acquired data.

5. Method, according to claim 4, characterized by further comprising the step of:
5) moving, manually or automatically, the hybrid probe along the length of the pipe, thus generating an aging profile of the same.
